(19) United States
(12) Patent Application Publication
(10) Pub. No.: US 2026/0027967 A1
(43) Pub. Date: Jan. 29, 2026

(54) NOTIFICATION CONTROL APPARATUS, NOTIFICATION CONTROL METHOD, AND STORAGE MEDIUM

(71) Applicant: HONDA MOTOR CO., LTD., Tokyo (JP)

(72) Inventors: Satoru Shinkawa, Wako-shi (JP); Shigenobu Mitsuzawa, Wako-shi (JP); Hiroshi Ono, Wako-shi (JP); Hiroki Taniguchi, Wako-shi (JP); Nozomi Nagatani, Wako-shi (JP)

(21) Appl. No.: 19/213,119

(22) Filed: May 20, 2025

(30) Foreign Application Priority Data
Jul. 26, 2024 (JP) 2024-121013

(51) Int. Cl. B60Q 5/00; A61B 5/00; A61B 5/18; B60K 35/22; G10L 15/22

(52) U.S. Cl. CPC B60Q 5/005; A61B 5/18; A61B 5/7415; A61B 5/746; B60K 35/22; G10L 15/22; B60K 2360/178

(57) ABSTRACT

A smartphone includes: a first biological information acquisition unit that acquires first biological information being biological information about a driver while driving a vehicle; a driving behavior acquisition unit that acquires driving behavior information indicating driving behavior of the driver; a danger level calculation unit that calculates a driving danger level indicating a danger degree while the driver is driving the vehicle, based on the first biological information and the driving behavior information; a speech output unit that performs speech output for the driver, in accordance with a degree of change in the driving danger level; a response acquisition unit that acquires response information indicating a response from the driver to the speech output; and an output control unit that controls the speech output unit based on the response information.

### INCORPORATION BY REFERENCE

The present application claims priority under 35 U.S.C. § 119 to Japanese Patent Application No. 2024-121013 filed on Jul. 26, 2024. The content of the application is incorporated herein by reference in its entirety.

### BACKGROUND OF THE INVENTION

### Field of the Invention

The present invention relates to a notification control apparatus, a notification control method, and a storage medium.

### Description of the Related Art

There has been proposed an apparatus that acquires biological information about a driver and driving information relating to a driving operation of the driver, and determines appropriateness of the driver's driving based on these acquired pieces of information (for example, see Japanese Patent Laid-Open No. 2023-145468).

In recent years, efforts have gained momentum for providing access to sustainable transport systems that also take into account people in vulnerable situations among traffic participants. Towards this end, efforts are focused on research and development for further improving traffic safety, convenience, and the like through research and development relating to a driver assistance system that supports the safety and health of drivers.

However, in the driver assistance system, a problem is to further improve traffic safety, convenience, and the like by outputting speech in accordance with a health state and driving behavior of a driver, and controlling speech output based on a response from the driver to the speech output.

In order to solve the above problem, an object of the present application is to output speech in accordance with a health state and driving behavior of a driver, and to control speech output based on a response from the driver to the speech output. Thus, the present application contributes to development of a sustainable transportation system.

### SUMMARY OF THE INVENTION

According to a first aspect for achieving the above object, a notification control apparatus includes: a first biological information acquisition unit that acquires first biological information being biological information about a driver while driving a vehicle; a driving behavior acquisition unit that acquires driving behavior information indicating driving behavior of the driver; a danger level calculation unit that calculates a driving danger level indicating a danger degree while the driver is driving the vehicle, based on the first biological information and the driving behavior information; a speech output unit that performs speech output for the driver, in accordance with a degree of change in the driving danger level; a response acquisition unit that acquires response information indicating a response from the driver to the speech output; and an output control unit that controls the speech output unit based on the response information.

According to a second aspect for achieving the above object, a notification control method includes: a first acquisition step of acquiring first biological information being biological information about a driver while driving a vehicle; a second acquisition step of acquiring driving behavior information indicating driving behavior of the driver; a calculation step of calculating a driving danger level indicating a danger degree while the driver is driving the vehicle, based on the first biological information and the driving behavior information; an output step of performing speech output for the driver, in accordance with a degree of change in the driving danger level; a third acquisition step of acquiring response information indicating a response from the driver to the speech output; and an output control step of controlling the speech output, based on the response information.

According to a third aspect for achieving the above object, a non-transitory computer readable storage medium storing a notification control program causes a processor included in a notification control apparatus to function as: a first biological information acquisition unit configured to acquire first biological information being biological information about a driver while driving a vehicle; a driving behavior acquisition unit configured to acquire driving behavior information indicating driving behavior of the driver; a danger level calculation unit configured to calculate a driving danger level indicating a danger degree while the driver is driving the vehicle, based on the first biological information and the driving behavior information; a speech output unit configured to perform speech output for the driver, in accordance with a degree of change in the driving danger level; a response acquisition unit configured to acquire response information indicating a response from the driver to the speech output; and an output control unit configured to control the speech output unit, based on the response information.

According to the notification control apparatus, the notification control method, and the storage medium, it is possible to output speech in accordance with a health state and driving behavior of a driver, and to appropriately control speech output based on a response from the driver to the speech output.

### BRIEF DESCRIPTION OF THE DRAWINGS

FIG. 1 is a diagram showing an example of a configuration of a notification control system;

FIG. 2 is a diagram showing an example of a configuration of a smartwatch;

FIG. 3 is a diagram showing an example of a configuration of an in-vehicle apparatus;

FIG. 4 is a diagram showing an example of the configuration of the in-vehicle apparatus;

FIG. 5 is a diagram showing an example of a configuration of a smartphone;

FIG. 6 is a diagram showing an example of correlation between biological information, a health rating, and advice information; and

FIG. 7 is a flowchart showing an example of processing of the smartphone.

### DETAILED DESCRIPTION OF THE INVENTION

Hereinafter, embodiments of a notification control apparatus, a notification control method, and a notification control program will be described with reference to the drawings.

### 1. Configuration of Notification Control System

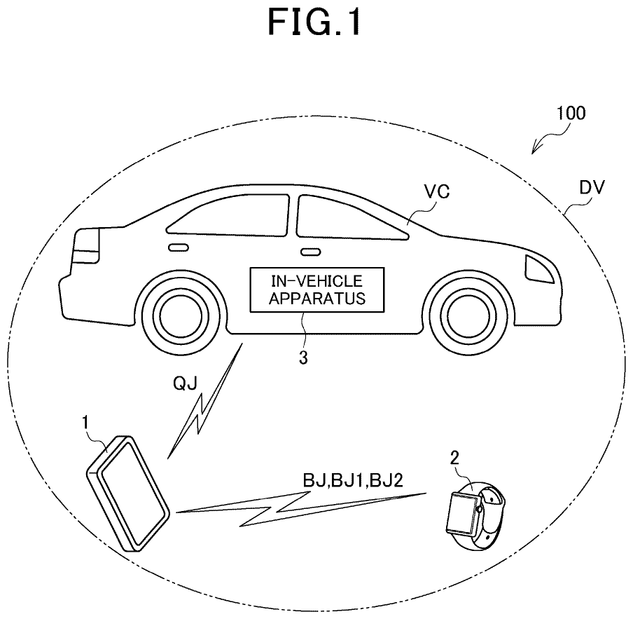

First, a configuration of a notification control system 100 will be described with reference to FIG. 1. FIG. 1 is a diagram showing an example of the configuration of the notification control system 100. The notification control system 100 corresponds to an example of the above “driver assistance system”.

As shown in FIG. 1, the notification control system 100 includes a smartphone 1, a smartwatch 2, and an in-vehicle apparatus 3. Note that a dash-dotted line in FIG. 1 indicates that the smartphone 1, the smartwatch 2, and a vehicle VC are property of a driver DV. The driver DV drives the vehicle VC.

The smartphone 1 wirelessly communicates with the smartwatch 2 and the in-vehicle apparatus 3 in accordance with, for example, a communication standard such as Wi-Fi®.

Note that the smartphone 1 may wirelessly communicate with the smartwatch 2 and the in-vehicle apparatus 3 through, for example, a communication method supporting a communication standard such as Bluetooth®, Bluetooth Low Energy (BLE), Wi-Fi®, or Zigbee®.

The smartphone 1 acquires biological information BJ about the driver DV from the smartwatch 2. The smartphone 1 acquires driving behavior information QJ from the in-vehicle apparatus 3.

The smartphone 1 calculates a driving danger level DR indicating a danger degree while the driver DV is driving the vehicle VC, based on the biological information BJ and the driving behavior information QJ. The smartphone 1 performs speech output in accordance with a change in the driving danger level DR. For example, the smartphone 1 outputs, as the speech output, advice information AD for the driver DV using speech. The smartphone 1 further controls the speech output based on a response from the driver DV to the speech output.

The smartphone 1 corresponds to an example of the “notification control apparatus”.

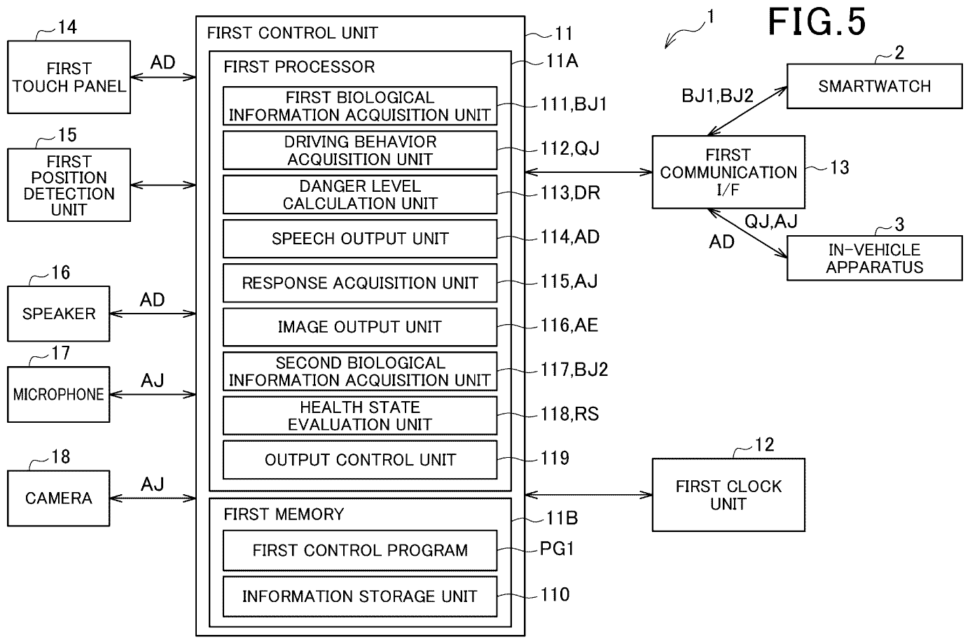

The smartphone 1 will be described further with reference to FIGS. 5 to 7.

The smartwatch 2 is worn on a wrist of the driver DV. The smartwatch 2 generates first biological information BJ1. The first biological information BJ1 includes, for example, a heart rate of the driver DV.

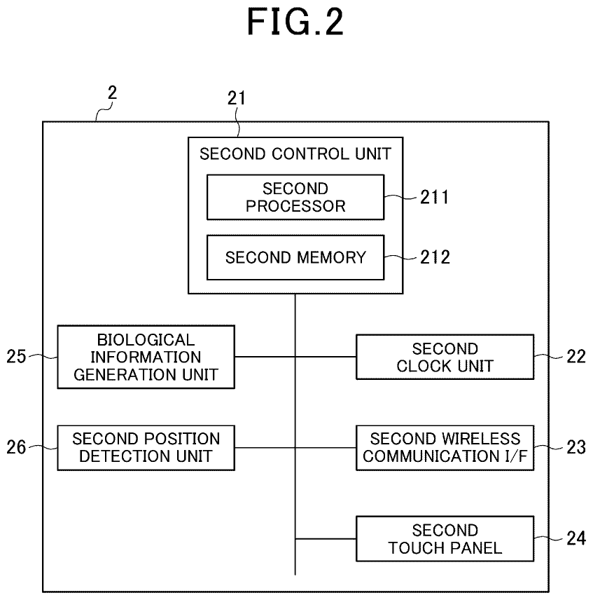

The smartwatch 2 will be described further with reference to FIG. 2.

The in-vehicle apparatus 3 is installed in the vehicle VC. The in-vehicle apparatus 3 generates the driving behavior information QJ. The driving behavior information QJ includes operation information from when the driver DV has driven the vehicle VC.

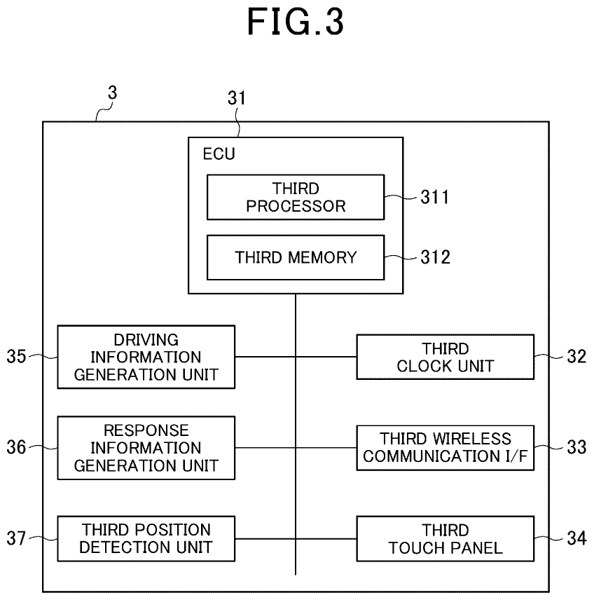

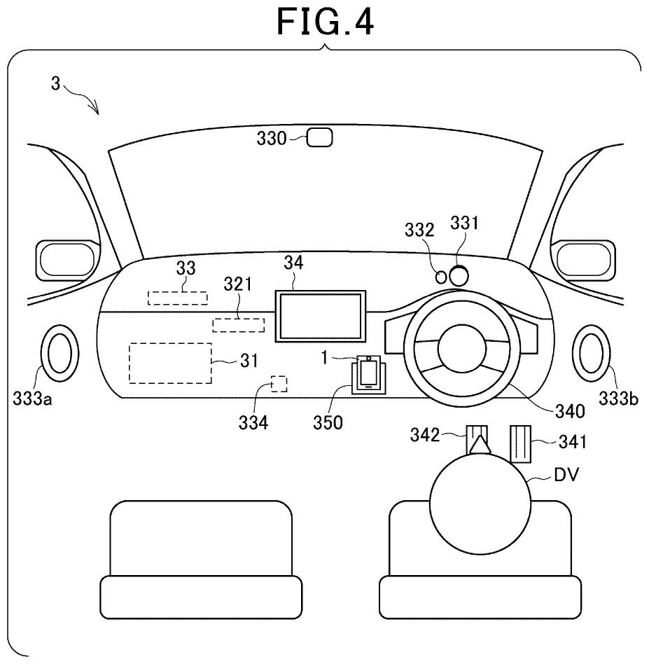

The in-vehicle apparatus 3 will be described further with reference to FIGS. 3 and 4.

### 2. Configuration of Smartwatch

Next, a configuration of the smartwatch 2 will be described with reference to FIG. 2. FIG. 2 is a diagram showing an example of the configuration of the smartwatch 2.

As shown in FIG. 2, the smartwatch 2 includes a second control unit 21, a second clock unit 22, a second wireless communication interface 23, a second touch panel 24, a biological information generation unit 25, and a second position detection unit 26.

The second control unit 21 controls each unit of the smartwatch 2. The second control unit 21 includes a second processor 211 and a second memory 212.

The second memory 212 consists of, for example, a non-volatile semiconductor memory, or volatile and non-volatile semiconductor memories. The second processor 211 consists of a central processing unit (CPU), and microprocessor unit (MPU), or the like. The second processor 211 may consist of a single processor or can also consist of a plurality of processors.

The second processor 211 controls each unit of the smartwatch 2 by executing a second control program stored in the second memory 212.

The second clock unit 22 has a clock function of measuring a date and time. The second clock unit 22 acquires date and time information from a server apparatus (not illustrated). The second clock unit 22 adjusts the clock function by using the acquired date and time information. The second clock unit 22 displays the date and time information on the second touch panel 24.

The second wireless communication interface 23 includes an antenna that receives a wireless signal and an interface circuit that processes the wireless signal received by the antenna. The second wireless communication interface 23 performs wireless communication with external apparatuses including the server apparatus (not illustrated) and the smartphone 1.

The second touch panel 24 includes a display panel and a touch sensor. The display panel consists of a liquid crystal panel, an organic electroluminescent (EL) panel, or the like. The touch sensor detects a touch operation of the driver DV on the display panel.

The second touch panel 24 displays an image on the display panel in accordance with an instruction from the second control unit 21. The second touch panel 24 detects a touch operation of the driver DV on the display panel.

The biological information generation unit 25 is a computer (ECU) including a processor and a memory, and generates the biological information BJ.

The biological information generation unit 25 measures, at preset intervals, the biological information BJ that is the biological information about the driver DV who is wearing the smartwatch 2.

The biological information BJ includes first biological information BJ1 and second biological information BJ2. The first biological information BJ1 is biological information about the driver DV while the driver DV is driving the vehicle VC. The second biological information BJ2 is biological information about the driver DV in a first period before the driver DV starts driving.

The first biological information BJ1 includes, for example, first sleep information BJ11, first exercise information BJ12, and first stress information BJ13. The second biological information BJ2 includes, for example, second sleep information BJ21, second exercise information BJ22, and second stress information BJ23.

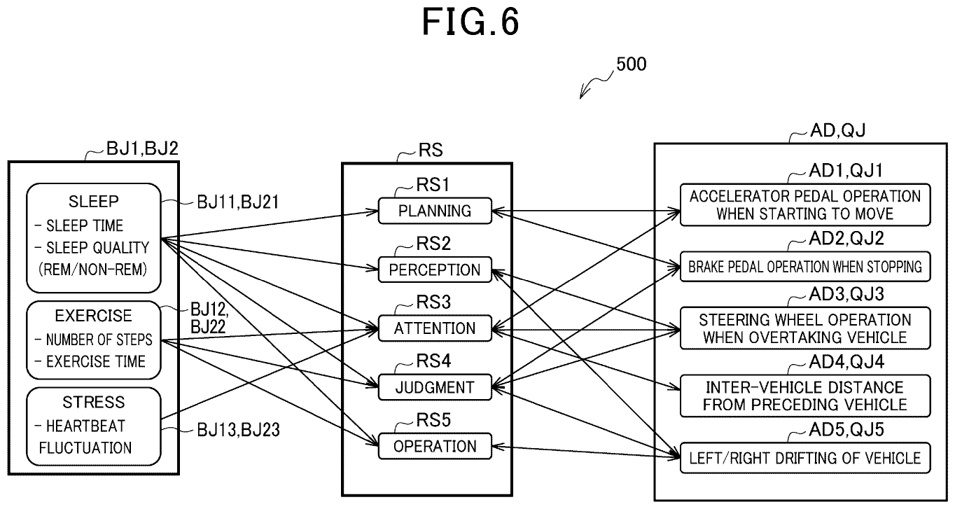

The first sleep information BJ11, the first exercise information BJ12, the first stress information BJ13, the second sleep information BJ21, the second exercise information BJ22, and the second stress information BJ23 will be described further with reference to FIGS. 5 and 6.

The biological information generation unit 25 outputs the measured biological information BJ to the second control unit 21. The second control unit 21 transmits the biological information BJ to the smartphone 1 via the second wireless communication interface 23.

The second position detection unit 26 includes a global navigation satellite system (GNSS) sensor. The second position detection unit 26 measures a position, that is, a latitude, a longitude, an altitude, and the like of the smartwatch 2, by receiving a positioning signal transmitted from a GNSS satellite. The second position detection unit 26 outputs position information indicating the measured position to the second control unit 21.

### 3. Configuration of In-Vehicle Apparatus

Next, a configuration of the in-vehicle apparatus 3 will be described with reference to FIGS. 3 and 4. FIG. 3 is a diagram showing an example of the configuration of the in-vehicle apparatus 3.

As shown in FIG. 3, the in-vehicle apparatus 3 includes an electronic control unit (ECU) 31, a third clock unit 32, a third wireless communication interface 33, a third touch panel 34, a driving information generation unit 35, a response information generation unit 36, and a third position detection unit 37.

The ECU 31 controls each unit of the in-vehicle apparatus 3. The ECU 31 includes a third processor 311 and a third memory 312.

The third memory 312 consists of, for example, a non-volatile semiconductor memory, volatile and non-volatile semiconductor memories, or the like. The third processor 311 consists of a CPU, an MPU, or the like. The third processor 311 may consist of a single processor or can also consist of a plurality of processors.

The third processor 311 controls each unit of the in-vehicle apparatus 3 by executing a third control program stored in the third memory 312.

The third clock unit 32 has a clock function of measuring the date and time. The third clock unit 32 acquires the date and time information from the server apparatus (not illustrated). The third clock unit 32 adjusts the clock function by using the acquired date and time information. The third clock unit 32 displays the date and time information on the third touch panel 34.

The third wireless communication interface 33 includes an antenna that receives a wireless signal and an interface circuit that processes the wireless signal received by the antenna. The third wireless communication interface 33 performs wireless communication with external apparatuses including the server apparatus (not illustrated) and the smartphone 1.

The third touch panel 34 includes a display panel and a touch sensor. The display panel consists of a liquid crystal panel, an organic EL panel, or the like. The touch sensor detects a touch operation of the driver DV on the display panel.

The third touch panel 34 displays an image on the display panel in accordance with an instruction from the ECU 31. The third touch panel 34 detects a touch operation of the driver DV on the display panel.

The driving information generation unit 35 is a computer (ECU) including a processor and a memory, and generates the driving behavior information QJ.

The driving information generation unit 35 acquires the driving behavior information QJ from various sensors disposed in the vehicle VC. The driving behavior information QJ includes, for example, vehicle speed information, acceleration information, accelerator operation information, brake operation information, and steering wheel operation information.

Note that the driving behavior information QJ may include road information about a road on which the vehicle VC travels. The road information includes, for example, construction information or traffic congestion information.

The vehicle speed information indicates a travel speed of the vehicle VC. The acceleration information indicates an acceleration detected by an acceleration sensor. The acceleration sensor detects, for example, an acceleration in an up-down direction, an acceleration in a front-rear direction, and an acceleration in a left-right direction of the vehicle VC.

The accelerator operation information indicates an operation state of an acceleration pedal 341 caused by the driver DV. The accelerator operation information includes, for example, a pressed amount and a pressing speed of the acceleration pedal 341.

The brake operation information indicates an operation state of a brake pedal 342 caused by the driver DV. The brake operation information includes, for example, a pressed amount and a pressing speed of the brake pedal 342.

The steering wheel operation information indicates an operation state of a steering wheel 340 caused by the driver DV. The steering wheel operation information includes, for example, a steering angle and a change speed of the steering angle of the steering wheel 340.

The acceleration pedal 341, the brake pedal 342, and the steering wheel 340 will be described further with reference to FIG. 4.

The driving information generation unit 35 outputs the driving behavior information QJ acquired from the various sensors to the ECU 31. The ECU 31 transmits the driving behavior information QJ to the smartphone 1 via the third wireless communication interface 33.

The response information generation unit 36 is a computer (ECU) including a processor and a memory, and acquires response information AJ from various sensors disposed in the vehicle VC. The response information AJ indicates the response from the driver DV to the speech output for the driver DV.

The response information generation unit 36 acquires the response information AJ from the various sensors disposed in the vehicle VC. The response information AJ includes, for example, driver image information and driver speech information.

The driver image information indicates an image of the driver DV. The image of the driver DV is generated by a driver camera 331.

The driver speech information indicates speech produced by the driver DV. The speech produced by the driver DV is acquired by a driver microphone 332.

The driver camera 331 and the driver microphone 332 will be described further with reference to FIG. 4.

The response information generation unit 36 outputs the response information AJ acquired from the various sensors to the ECU 31. The ECU 31 transmits the response information AJ to the smartphone 1 via the third wireless communication interface 33.

The third position detection unit 37 includes a GNSS sensor. The third position detection unit 37 measures a position, that is, a latitude, a longitude, an altitude, and the like of the vehicle VC, by receiving a positioning signal transmitted from a GNSS satellite. The third position detection unit 37 outputs position information indicating the measured position to the ECU 31.

Next, the configuration of the in-vehicle apparatus 3 will be further described with reference to FIG. 4. FIG. 4 is a diagram showing an example of the configuration of the in-vehicle apparatus 3. FIG. 4 is a view when looking forward from a top portion direction of a driver seat in the vehicle VC.

As shown in FIG. 4, the steering wheel 340 described with reference to FIG. 3 is disposed, for example, in front of the driver DV. The acceleration pedal 341 and the brake pedal 342 described with reference to FIG. 3 are disposed, for example, at the feet of the driver DV. The third touch panel 34 described with reference to FIG. 3 is, for example, to the left of the steering wheel 340 and is disposed substantially at the center of a dashboard. The driver camera 331 and the driver microphone 332 described with reference to FIG. 3 are, for example, disposed in the dashboard behind (at a vehicle front side of) the steering wheel 340. The ECU 31 described with reference to FIG. 3 is disposed, for example, in the dashboard in front of a passenger seat. The third wireless communication interface 33 described with reference to FIG. 3 is, for example, disposed in the dashboard in front of the passenger seat.

Note that illustration of the third clock unit 32, the driving information generation unit 35, the response information generation unit 36, and the third position detection unit 37 described with reference to FIG. 3 is omitted in FIG. 4.

The driver camera 331 includes an image sensor such as a charge-coupled device (CCD) or a complementary metal-oxide-semiconductor (CMOS). The driver camera 331 images the driver DV and generates a captured image, or the image of the driver. The image of the driver includes, for example, at least a head portion of the driver. The image of the driver preferably includes, for example, both hands of the driver.

The driver camera 331 outputs the generated image of the driver to the ECU 31.

The driver camera 331 constitutes a portion of the response information generation unit 36.

The image of the driver corresponds to an example of the response information AJ.

As shown in FIG. 4, the in-vehicle apparatus 3 further includes a navigation apparatus 321, a front camera 330, a left speaker 333a, a right speaker 333b, an acceleration sensor 334, and a holder 350.

The navigation apparatus 321 includes the third position detection unit 37 described in reference with FIG. 3 and displays, for example, the position on a map of the vehicle VC on the third touch panel 34. The navigation apparatus 321 is disposed in the dashboard in front of the passenger seat.

The front camera 330 captures a front view of the vehicle VC. The front camera 330 is disposed on an inner side of a top portion of a front window.

The left speaker 333a is disposed on an inner side of a left door of the vehicle VC. The right speaker 333b is disposed on an inner side of a right door of the vehicle VC. The left speaker 333a and the right speaker 333b output speech. For example, the left speaker 333a and the right speaker 333b may output speech indicating advice information AD.

The acceleration sensor 334 detects, for example, the acceleration in the up-down direction, the front-rear direction, and the left-right direction of the vehicle VC. The acceleration sensor 334 is disposed, for example, inside the dashboard.

The holder 350 fixably supports the smartphone 1 to the dashboard. When the driver DV is driving the vehicle VC, the driver DV places the smartphone 1 in the holder 350. The holder 350 is disposed, for example, on the dashboard in front of the driver seat to the left.

### 4. Configuration of Smartphone

Next, a configuration of the smartphone 1 will be described with reference to FIG. 5. FIG. 5 is a diagram showing an example of the configuration of the smartphone 1.

The smartphone 1 includes a first control unit 11, a first clock unit 12, a first wireless communication interface 13, a first touch panel 14, a first position detection unit 15, a speaker 16, a microphone 17, and a camera 18.

The first control unit 11 controls each unit of the smartphone 1. The first control unit 11 includes a first processor 11A and a first memory 11B.

The first memory 11B consists of, for example, a non-volatile semiconductor memory, or volatile and non-volatile semiconductor memories. The first processor 11A consists of a CPU, an MPU, or the like. The first processor 11A may consist of a single processor or can also consist of a plurality of processors.

The first processor 11A controls each unit of the smartphone 1 by executing a first control program PG1 stored in the first memory 11B.

The first clock unit 12 has a clock function of measuring the date and time. The first clock unit 12 acquires the date and time information from the server apparatus (not illustrated). The first clock unit 12 adjusts the clock function by using the acquired date and time information. The first clock unit 12 displays the date and time information on the first touch panel 14.

The first wireless communication interface 13 includes an antenna that receives a wireless signal and an interface circuit that processes the wireless signal received by the antenna. The first wireless communication interface 13 performs wireless communication with external apparatuses including the server apparatus (not illustrated), the smartwatch 2, and the in-vehicle apparatus 3.

The first touch panel 14 includes a display panel and a touch sensor. The display panel consists of a liquid crystal panel, an organic EL panel, or the like. The touch sensor detects a touch operation of the driver DV on the display panel.

The first touch panel 14 displays an image on the display panel in accordance with an instruction from the first control unit 11. The first touch panel 14 displays, for example, an image indicating the advice information AD on the display panel in accordance with an instruction from the first control unit 11.

The first touch panel 14 detects a touch operation of the driver DV on the display panel.

The first position detection unit 15 includes a GNSS sensor. The first position detection unit 15 measures a position, that is, a latitude, a longitude, an altitude, and the like of the smartphone 1, by receiving a positioning signal transmitted from a GNSS satellite. The first position detection unit 15 outputs position information indicating the measured position to the first control unit 11.

The speaker 16 outputs various speech in accordance with an instruction from the first control unit 11. For example, the speaker 16 outputs speech indicating the advice information AD in accordance with an instruction from the first control unit 11.

The output of the speech indicating the advice information AD corresponds to an example of the “speech output”.

The microphone 17 accepts an input of various speech in accordance with an instruction from the first control unit 11. The microphone 17 outputs the accepted speech to the first control unit 11.

The microphone 17 may accept, for example, speech from the driver DV.

The speech of the driver DV corresponds to an example of the response information AJ.

The camera 18 captures various images in accordance with an instruction from the first control unit 11. The camera 18 includes an image sensor such as a CCD or a CMOS. The camera 18 outputs the generated images to the first control unit 11.

The camera 18 may, for example, image the driver DV and generate a captured image.

The captured image of the driver DV corresponds to an example of the response information AJ.

The first control unit 11 includes a first biological information acquisition unit 111, a driving behavior acquisition unit 112, a danger level calculation unit 113, a speech output unit 114, a response acquisition unit 115, an image output unit 116, a second biological information acquisition unit 117, a health state evaluation unit 118, an output control unit 119, and an information storage unit 110.

To be specific, the first processor 11A functions as the first biological information acquisition unit 111, the driving behavior acquisition unit 112, the danger level calculation unit 113, the speech output unit 114, the response acquisition unit 115, the image output unit 116, the second biological information acquisition unit 117, the health state evaluation unit 118, and the output control unit 119 by executing the first control program PG1 stored in the first memory 11B. The first processor 11A causes the first memory 11B to function as the information storage unit 110 by executing the first control program PG1 stored in the first memory 11B.

The first processor 11A corresponds to an example of the “processor”.

The first control program PG1 corresponds to an example of the “notification control program”.

The information storage unit 110 stores the biological information BJ and the driving behavior information QJ. The biological information BJ includes the first biological information BJ1 and the second biological information BJ2.

The first biological information BJ1 is, for example, acquired from the smartwatch 2 by the first biological information acquisition unit 111, and stored in the information storage unit 110 by the first biological information acquisition unit 111.

The second biological information BJ2 is, for example, acquired from the smartwatch 2 by the second biological information acquisition unit 117, and stored in the information storage unit 110 by the second biological information acquisition unit 117.

The driving behavior information QJ is, for example, acquired from the in-vehicle apparatus 3 by the driving behavior acquisition unit 112, and stored in the information storage unit 110 by the driving behavior acquisition unit 112.

The first biological information acquisition unit 111 acquires, for example, the first biological information BJ1. The first biological information BJ1 is the biological information about the driver DV while driving the vehicle VC. While driving the vehicle VC indicates a period from when the driver DV starts driving the vehicle VC to when the driver DV stops driving the vehicle VC. The first biological information acquisition unit 111 acquires, for example, the first biological information BJ1 from the smartwatch 2. The first biological information BJ1 includes, for example, the first sleep information BJ11, the first exercise information BJ12, and the first stress information BJ13.

The first sleep information BJ11 indicates, for example, a drowsiness degree estimated from the second sleep information BJ21 to be described below. The first exercise information BJ12 indicates, for example, an exercise ability level estimated from the second exercise information BJ22 to be described below. The first stress information BJ13 indicates a stress degree experienced by the driver DV. The first stress information BJ13 includes heartbeat information and heartbeat fluctuation information. The heartbeat information indicates, for example, a heart rate per minute. The heartbeat fluctuation information indicates a state of a change in the heart rate.

The driving behavior acquisition unit 112 acquires the driving behavior information QJ indicating the driving behavior of the driver DV. The driving behavior acquisition unit 112 acquires, for example, the driving behavior information QJ from the in-vehicle apparatus 3. The driving behavior information QJ includes, for example, the vehicle speed information, the acceleration information, the accelerator operation information, the brake operation information, and the steering wheel operation information.

The danger level calculation unit 113 calculates the driving danger level DR indicating the danger degree while the driver DV is driving the vehicle VC, based on the first biological information BJ1 and the driving behavior information QJ.

The danger level calculation unit 113 generates, for example, a reference driving property indicating a property of the driving behavior of the driver DV, based on a history of the driving behavior information QJ in past driving by the driver DV. The danger level calculation unit 113 evaluates a variability degree in the reference driving property of the driving behavior indicated by the driving behavior information QJ acquired by the driving behavior acquisition unit 112 in the current driving. The danger level calculation unit 113 calculates the driving danger level DR indicating the danger degree while the driver DV is driving the vehicle VC, based on the variability degree.

For example, a case will be described in which the driving behavior is an accelerator operation when the driver DV starts moving the vehicle VC.

The danger level calculation unit 113 generates, for example, a reference driving property indicating a property of the accelerator operation of the driver DV when the driver DV starts moving the vehicle VC, based on a history of the accelerator operation when starting to move the vehicle VC in past driving by the driver DV. The reference driving property is, for example, an acceleration distribution indicating a frequency distribution of an average value of an acceleration caused by the accelerator operation when the driver DV starts moving the vehicle VC.

Next, the danger level calculation unit 113 evaluates a variability degree in the acceleration distribution of the accelerator operation when the driver DV starts moving the vehicle VC indicated by the driving behavior information QJ acquired by the driving behavior acquisition unit 112 in the current driving.

For example, the danger level calculation unit 113 calculates the driving danger level DR as “0” when the acceleration caused by the accelerator operation when the driver DV starts moving the vehicle VC in the current driving is included in a range of a first threshold value with respect to the average value of the acceleration distribution. The first threshold value is, for example, two times a standard deviation.

Note that the worse the health state indicated by the first biological information BJ1 is, the more the danger level calculation unit 113 lowers the first threshold value.

For example, the danger level calculation unit 113 calculates the driving danger level DR as “1” when the acceleration caused by the accelerator operation when the driver DV starts moving the vehicle VC in the current driving is outside the range of the first threshold value of the acceleration distribution and is included in a range of a second threshold value of the acceleration distribution with respect to the average value of the acceleration distribution. The range of the second threshold value is, for example, three times the standard deviation.

For example, the danger level calculation unit 113 calculates the driving danger level DR as “2” when the acceleration caused by the accelerator operation in the current driving is outside the range of the second threshold value of the acceleration distribution with respect to the average value of the acceleration distribution.

Note that the worse the health state indicated by the first biological information BJ1 is, the more the danger level calculation unit 113 lowers the second threshold value.

In this way, the danger level calculation unit 113 calculates the driving danger level DR of the accelerator operation indicated by the driving behavior information QJ acquired in the current driving.

The speech output unit 114 performs the speech output for the driver DV in accordance with a degree of change in the driving danger level DR. The speech output unit 114 causes the speaker 16 to output, for example, the speech indicating the advice information AD for the driver DV.

Note that the speech output unit 114 may cause the left speaker 333a and the right speaker 333b of the vehicle VC to output the speech indicating the advice information AD for the driver DV. In this case, the speech output unit 114 instructs the ECU 31 of the vehicle VC to output the speech indicating the advice information AD for the driver DV to the left speaker 333a and the right speaker 333b of the vehicle VC.

The speech output unit 114 performs the speech output indicating the advice information AD at a predetermined frequency, in accordance with the degree of change in the driving danger level DR and a travel route traveled by the vehicle VC.

Note that the speech output unit 114 acquires, for example, the travel route traveled by the vehicle VC from the navigation apparatus 321 via the ECU 31 of the vehicle VC.

The speech output unit 114 calculates, for example, an integrated value of the driving danger level DR after the driver DV starts driving. The speech output unit 114 performs the speech output indicating the advice information AD for the driver DV, when the integrated value of the driving danger level DR becomes equal to or higher than a third threshold value. The third threshold value is, for example, “3”.

Even when the integrated value of the driving danger level DR does not become equal to or higher than the third threshold value, the speech output unit 114 performs the speech output indicating the advice information AD, when an intersection or the like is present where accidents frequently occur in a movement direction of the vehicle VC on the travel route traveled by the vehicle VC.

The response acquisition unit 115 acquires the response information AJ indicating the response from the driver DV to the speech output. The response information AJ includes, for example, the driver image information and the driver speech information. The response acquisition unit 115 acquires the response information AJ from, for example, the in-vehicle apparatus 3.

The output control unit 119 controls the speech output unit 114 based on the response information AJ.

For example, when the response information AJ acquired by the response acquisition unit 115 indicates an unpleasant emotion of the driver DV, the output control unit 119 instructs the speech output unit 114 to stop the speech output indicating the advice information AD or lower the frequency of performing the speech output indicating the advice information AD.

The response information AJ indicating an unpleasant emotion of the driver DV is, for example, when it is estimated from the driver image information that the driver has an unpleasant facial expression. The response information AJ indicating an unpleasant emotion of the driver DV is, for example, when it is estimated from the driver speech information that the driver is making a statement indicating an unpleasant emotion.

For example, when the response information AJ acquired by the response acquisition unit 115 indicates an unpleasant emotion of the driver DV, the output control unit 119 instructs the image output unit 116 to perform image output for the driver DV. For example, the output control unit 119 instructs the image output unit 116 to perform the image output indicating the advice information AD for the driver DV.

The image output unit 116 performs the image output indicating the advice information AD for the driver DV, in accordance with the degree of change in the driving danger level DR. The image output unit 116 performs, for example, the image output indicating the advice information AD for the driver DV on the first touch panel 14. The image output unit 116 may, for example, perform the image output indicating the advice information AD for the driver DV on the third touch panel 34 of the vehicle VC. In this case, the image output unit 116 instructs the ECU 31 of the vehicle VC to perform the image output indicating the advice information AD for the driver DV on the third touch panel 34 of the vehicle VC.

The second biological information acquisition unit 117 acquires the second biological information BJ2. The second biological information BJ2 is biological information about the driver DV before the driver DV starts driving the vehicle VC. The second biological information acquisition unit 117 acquires, for example, the second biological information BJ2 from the smartwatch 2.

The second biological information BJ2 includes the second sleep information BJ21, the second exercise information BJ22, and the second stress information BJ23.

The second sleep information BJ21 includes, for example, sleep time information and information indicating sleep quality. The sleep time information indicates, for example, a sleep time per day of the driver DV. The information indicating sleep quality includes, for example, REM sleep time information and non-REM sleep time information per day of the driver DV.

The second exercise information BJ22 includes, for example, step number information and exercise time information. The step number information indicates, for example, a cumulative value of the number of steps per day of the driver DV. The exercise time information indicates, for example, a cumulative value of an exercise time per day of the driver DV.

The second stress information BJ23 includes heartbeat information and heartbeat fluctuation information. The heartbeat information indicates, for example, a heart rate per minute. The heartbeat fluctuation information indicates a state of a change in the heart rate.

The health state evaluation unit 118 evaluates the health state of the driver DV and generates a health rating RS indicating a healthiness degree of the driver DV, based on the second biological information BJ2. The health rating RS corresponds to each of five abilities indicating a driving ability AB of the driver DV. The driving ability AB consists of, for example, a planning ability AB1, a perception ability AB2, an attention ability AB3, a judgment ability AB4, and an operation ability AB5.

The health rating RS consists of a planning ability rating RS1, a perception ability rating RS2, an attention ability rating RS3, a judgment ability rating RS4, and an operation ability rating RS5.

The planning ability rating RS1 indicates the health rating RS corresponding to the planning ability AB1. The planning ability AB1 is, for example, an ability of the driver DV to make a driving plan. The driving plan is, for example, a plan to cause the vehicle VC to travel from a parking position to a destination.

The perception ability rating RS2 indicates the health rating RS corresponding to the perception ability AB2. The perception ability AB2 is, for example, an ability to perceive a surrounding situation while the driver DV is driving.

The attention ability rating RS3 indicates the health rating RS corresponding to the attention ability AB3. The attention ability AB3 is, for example, an ability to pay attention to the surrounding situation in order to anticipate danger when the driver DV is driving.

The judgment ability rating RS4 indicates the health rating RS corresponding to the judgment ability AB4. The judgment ability AB4 is, for example, an ability to judge whether to perform any type of operation in accordance with the surrounding situation while the driver DV is driving.

The operation ability rating RS5 indicates the health rating RS corresponding to the operation ability AB5. The operation ability AB5 is, for example, an ability to perform an appropriate driving operation in accordance with the surrounding situation while the driver DV is driving.

The speech output unit 114 performs the speech output based on the health rating RS and the degree of change in the driving danger level DR. The speech output indicates, for example, outputting the advice information AD using speech.

A correlation between the health rating RS and the advice information AD will be described further with reference to FIG. 6.

The output control unit 119 causes the speech output unit 114 to adjust the frequency of performing the speech output.

For example, the output control unit 119 causes the speech output unit 114 to increase the frequency of performing the speech output, when the health rating RS is equal to or higher than a predetermined value, and after the driving behavior acquisition unit 112 acquires the driving behavior information QJ increasing the driving danger level DR, and then the driving behavior acquisition unit 112 acquires, in a first period, the driving behavior information QJ indicating that the driving behavior has not been improved. The speech output indicates outputting the speech indicating the advice information AD for the driver DV.

The health rating RS consists of the planning ability rating RS1, the perception ability rating RS2, the attention ability rating RS3, the judgment ability rating RS4, and the operation ability rating RS5. The health rating RS being equal to or higher than the predetermined value indicates that, for example, all of the planning ability rating RS1, the perception ability rating RS2, the attention ability rating RS3, the judgment ability rating RS4, and the operation ability rating RS5 are equal to or higher than the predetermined value.

The health state evaluation unit 118 attaches, for example, a score out of ten to each of the planning ability rating RS1, the perception ability rating RS2, the attention ability rating RS3, the judgment ability rating RS4, and the operation ability rating RS5. In this case, the predetermined value is, for example, a score of “7”.

The driving behavior information QJ increasing the driving danger level DR indicates, for example, abruptly starting to move, abruptly braking, abruptly steering, an operation causing an inter-vehicle distance to fall below an appropriate value, an operation causing drifting of the vehicle VC, and the like.

The driving behavior information QJ indicating that the driving behavior has not been improved is, for example, the driving behavior information QJ increasing the driving danger level DR.

The first period is, for example, “ten minutes”.

For example, the output control unit 119 causes the speech output unit 114 to decrease the frequency of performing the speech output, when the health rating RS is equal to or higher than the predetermined value, and after the driving behavior acquisition unit 112 acquires the driving behavior information QJ increasing the driving danger level DR, and then the driving behavior acquisition unit 112 acquires, in a second period, the driving behavior information QJ indicating that the driving behavior has been improved. The speech output indicates outputting the speech indicating the advice information AD for the driver DV.

The driving behavior information QJ indicating that the driving behavior has been improved is, for example, the driving behavior information QJ not increasing the driving danger level DR.

The second period is, for example, “twenty minutes”.

For example, when the response information AJ acquired by the response acquisition unit 115 indicates an unpleasant emotion of the driver DV and the driving behavior acquisition unit 112 acquires, in a third period, the driving behavior information QJ indicating that the driving behavior has not been improved, the output control unit 119 instructs the speech output unit 114 to perform speech output of a matter that is not directly related to a driving operation of the driver DV. The speech output indicates outputting the speech indicating the advice information AD for the driver DV.

The matter that is not directly related to a driving operation of the driver DV corresponds to the content indicated by the advice information AD. The advice information AD of a matter that is not directly related to a driving operation of the driver DV is, for example, advice information AD indicating “Please take a break.” When a passenger capable of driving is present in the vehicle VC, the advice information AD of a matter that is not directly related to a driving operation of the driver DV is, for example, advice information AD indicating “Please allow your passenger to drive instead.”

The third period is, for example, “ten minutes”.

For example, the output control unit 119 instructs the speech output unit 114 to perform speech output with different content between when the driving danger level DR is increased based on the first biological information BJ1 and when the driving danger level DR is increased based on the driving behavior information QJ.

The driving danger level DR being increased based on the first biological information BJ1 is, for example, when a heart rate of the driver DV exceeds a normal range. The normal range is, for example, set based on a history of the heart rate of the driver DV.

When the heart rate of the driver DV exceeds the normal range as an example of the driving danger level DR being increased based on the first biological information BJ1, the speech output unit 114 performs, for example, the speech output indicating advice information AD indicating “You seem to be feeling unwell. Please take a break.” or the like.

When abruptly starting to move as an example of the driving danger level DR being increased based on the driving behavior information QJ, the speech output unit 114 performs the speech output indicating advice information AD indicating “Abruptly starting to move has been detected. Please beware as this is dangerous.” or the like.

### 5. Correlation Between Biological Information, Health Rating, and Advice Information

Next, a correlative relationship between the biological information BJ, the health rating RS, and the advice information AD will be described with reference to FIG. 6. FIG. 6 is a diagram showing an example of a correlation between the biological information BJ, the health rating RS, and the advice information AD. The health rating RS corresponds to each of the five abilities indicating the driving ability AB. FIG. 6 illustrates a correlation diagram 500.

The correlation diagram 500 includes the biological information BJ, the health rating RS, and the advice information AD.

As shown in FIG. 6, the biological information BJ includes the first biological information BJ1 and the second biological information BJ2.

The first biological information BJ1 is biological information about the driver DV when the driver DV is driving. The first biological information BJ1 includes, for example, the first sleep information BJ11, the first exercise information BJ12, and the first stress information BJ13.

The second biological information BJ2 is biological information about the driver DV before the driver DV starts driving. The second biological information BJ2 includes, for example, the second sleep information BJ21, the second exercise information BJ22, and the second stress information BJ23.

The health rating RS includes the planning ability rating RS1, the perception ability rating RS2, the attention ability rating RS3, the judgment ability rating RS4, and the operation ability rating RS5.

The health state evaluation unit 118 evaluates the health state of the driver DV and generates the health rating RS indicating the healthiness degree of the driver DV, based on the second biological information BJ2. The health rating RS consists of the planning ability rating RS1, the perception ability rating RS2, the attention ability rating RS3, the judgment ability rating RS4, and the operation ability rating RS5.

Note that the health state evaluation unit 118 may correct the health rating RS by using the first biological information BJ1.

Arrows between the first biological information BJ1 and the health rating RS in FIG. 6 indicate correlations.

For example, the planning ability rating RS1 correlates with the second sleep information BJ21. Accordingly, the health state evaluation unit 118 generates the planning ability rating RS1 based on the second sleep information BJ21.

For example, the perception ability rating RS2 correlates with the second sleep information BJ21. Accordingly, the health state evaluation unit 118 generates the perception ability rating RS2 based on the second sleep information BJ21.

For example, the attention ability rating RS3 correlates with the second sleep information BJ21, the second exercise information BJ22, and the second stress information BJ23. Accordingly, the health state evaluation unit 118 generates the attention ability rating RS3 based on the second sleep information BJ21, the second exercise information BJ22, and the second stress information BJ23.

For example, the judgment ability rating RS4 correlates with the second sleep information BJ21 and the second exercise information BJ22. Accordingly, the health state evaluation unit 118 generates the judgment ability rating RS4 based on the second sleep information BJ21 and the second exercise information BJ22.

For example, the operation ability rating RS5 correlates with the second sleep information BJ21 and the second exercise information BJ22. Accordingly, the health state evaluation unit 118 generates the operation ability rating RS5 based on the second sleep information BJ21 and the second exercise information BJ22.

As shown in FIG. 6, the advice information AD includes, for example, first advice information AD1, second advice information AD2, third advice information AD3, fourth advice information AD4, and fifth advice information AD5.

The driving behavior information QJ includes, as the driving behavior information QJ increasing the driving danger level DR, first behavior information QJ1, second behavior information QJ2, third behavior information QJ3, fourth behavior information QJ4, and fifth behavior information QJ5.

The first advice information AD1 is, for example, the advice information AD relating to an operation of the acceleration pedal 341 when the driver DV starts moving the vehicle VC. The first advice information AD1 is, for example, the advice information AD encouraging not to abruptly start moving.

The first behavior information QJ1 indicates an operation of the acceleration pedal 341 when the driver DV starts moving the vehicle VC. The speech output unit 114 causes, for example, the speaker 16 to output speech indicating the first advice information AD1, when the driving danger level DR is increased due to the first behavior information QJ1.

The second advice information AD2 is, for example, the advice information AD relating to an operation of the brake pedal 342 when the driver DV stops the vehicle VC. The second advice information AD2 is, for example, the advice information AD encouraging not to abruptly stop.

The second behavior information QJ2 indicates an operation of the brake pedal 342 when the driver DV stops the vehicle VC. The speech output unit 114 causes, for example, the speaker 16 to output speech indicating the second advice information AD2, when the driving danger level DR is increased due to the second behavior information QJ2.

The third advice information AD3 is, for example, the advice information AD relating to an operation of the steering wheel 340 when the driver DV causes the vehicle VC to overtake another vehicle. The third advice information AD3 is, for example, the advice information AD encouraging not to abruptly steer.

The third behavior information QJ3 indicates an operation of the steering wheel 340 when the driver DV causes the vehicle VC to overtake another vehicle. The speech output unit 114 causes, for example, the speaker 16 to output speech indicating the third advice information AD3, when the driving danger level DR is increased due to the third behavior information QJ3.

The fourth advice information AD4 is, for example, the advice information AD relating to an inter-vehicle distance between the vehicle VC that the driver DV is driving and another vehicle traveling in front. The fourth advice information AD4 is, for example, the advice information AD encouraging to keep an appropriate inter-vehicle distance.

The fourth behavior information QJ4 indicates an operation of changing the inter-vehicle distance between the vehicle VC that the driver DV is driving and another vehicle traveling in front. The fourth behavior information QJ4 includes, for example, an operation of the acceleration pedal 341 and an operation of the brake pedal 342. The speech output unit 114 causes, for example, the speaker 16 to output speech indicating the fourth advice information AD4, when the driving danger level DR is increased due to the fourth behavior information QJ4.

The fifth advice information AD5 is the advice information AD relating to drifting in the left-right direction of the vehicle VC that the driver DV is driving. The fifth advice information AD5 is, for example, the advice information AD encouraging to suppress drifting in the left-right direction of the vehicle VC.

The fifth behavior information QJ5 indicates an operation that causes drifting in the left-right direction of the vehicle VC that the driver DV is driving. The fifth behavior information QJ5 includes, for example, loosening of a grip state on the steering wheel 340. The speech output unit 114 causes, for example, the speaker 16 to output speech indicating the fifth advice information AD5, when the driving danger level DR is increased due to the fifth behavior information QJ5.

Arrows between the health rating RS and the advice information AD in FIG. 6 indicate correlations.

The speech output unit 114 selects, for example, the advice information AD corresponding to a rating with the lowest score from among the planning ability rating RS1, the perception ability rating RS2, the attention ability rating RS3, the judgment ability rating RS4, and the operation ability rating RS5.

For example, as shown in FIG. 6, the planning ability rating RS1 correlates with the first advice information AD1 and the second advice information AD2. Accordingly, the speech output unit 114 informs the driver DV of, for example, the first advice information AD1 and the second advice information AD2 with priority, when the planning ability rating RS1 is the rating with the lowest score.

The perception ability rating RS2 correlates with the third advice information AD3 and the fifth advice information AD5. Accordingly, the speech output unit 114 informs the driver DV of, for example, the third advice information AD3 and the fifth advice information AD5 with priority, when the perception ability rating RS2 is the rating with the lowest score.

The attention ability rating RS3 correlates with the first advice information AD1, the third advice information AD3, and the fourth advice information AD4. Accordingly, the speech output unit 114 informs the driver DV of, for example, the first advice information AD1, the third advice information AD3, and the fourth advice information AD4 with priority, when the attention ability rating RS3 is the rating with the lowest score.

The judgment ability rating RS4 correlates with the second advice information AD2, the third advice information AD3, and the fifth advice information AD5. Accordingly, the speech output unit 114 informs the driver DV of, for example, the second advice information AD2, the third advice information AD3, and the fifth advice information AD5 with priority, when the judgment ability rating RS4 is the rating with the lowest score.

The operation ability rating RS5 correlates with the fifth advice information AD5. Accordingly, the speech output unit 114 informs the driver DV of, for example, the fifth advice information AD5 with priority, when the operation ability rating RS5 is the rating with the lowest score.

### 6. Processing of Smartphone

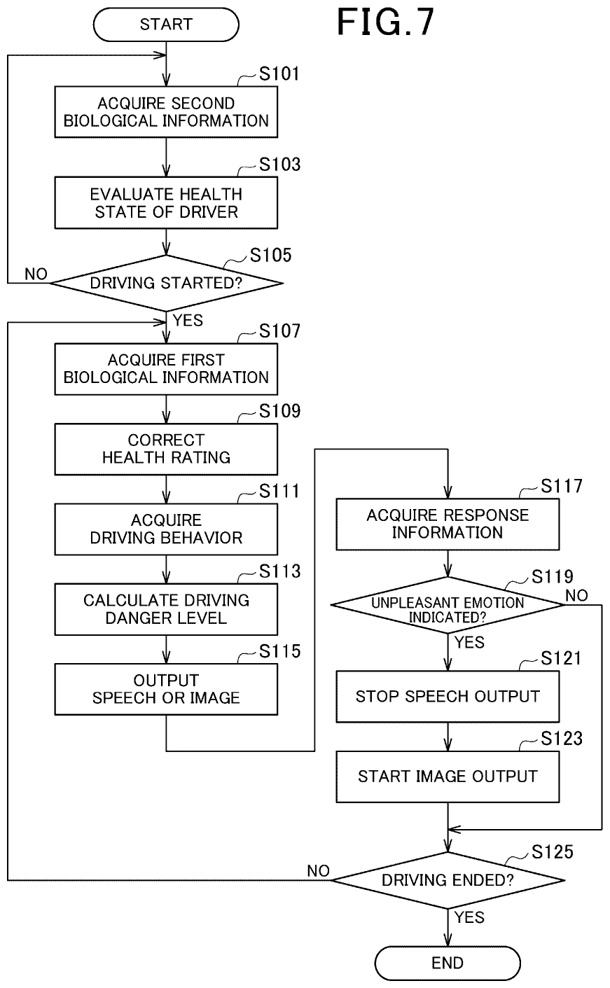

Next, processing of the smartphone 1 will be described with reference to FIG. 7. FIG. 6 is a flowchart showing an example of the processing of the smartphone 1.

First, in step S101, the second biological information acquisition unit 117 acquires, for example, the second biological information BJ2 from the smartwatch 2. The second biological information BJ2 is biological information about the driver DV before the driver DV starts driving the vehicle VC.

Next, in step S103, the health state evaluation unit 118 evaluates the health state of the driver DV and generates the health rating RS indicating the healthiness degree of the driver DV, based on the second biological information BJ2.

Next, in step S105, the first control unit 11 determines whether the driver DV has started driving the vehicle VC. The first control unit 11 determines, for example, that the driver DV has started driving the vehicle VC when the smartphone 1 is placed in the holder 350.

When the first control unit 11 determines that the driver DV has not started driving the vehicle VC (NO in step S105), the processing returns to step S101. When the first control unit 11 determines that the driver DV has started driving the vehicle VC (YES in step S105), the processing proceeds to step S107.

In step S107, the first biological information acquisition unit 111 acquires the first biological information BJ1 from the smartwatch 2.

Next, in step S109, the health state evaluation unit 118 corrects the health rating RS generated in step S103, based on the first biological information BJ1.

Next, in step S111, the driving behavior acquisition unit 112 acquires the driving behavior information QJ indicating the driving behavior of the driver DV from, for example, the in-vehicle apparatus 3.

Next, in step S113, the danger level calculation unit 113 calculates the driving danger level DR indicating the danger degree while the driver DV is driving the vehicle VC, based on the first biological information BJ1 and the driving behavior information QJ.

Next, in step S115, the speech output unit 114 performs the speech output indicating the advice information AD for the driver DV to the driver DV, in accordance with the degree of change in the driving danger level DR. For example, when the speech output from the speech output unit 114 is stopped, the image output unit 116 performs the image output indicating the advice information AD for the driver DV to the driver DV, in accordance with the degree of change in the driving danger level DR.

Hereinafter, a case will be described in which the speech output unit 114 performs the speech output indicating the advice information AD for the driver DV, in accordance with the degree of change in the driving danger level DR.

Next, in step S117, the response acquisition unit 115 acquires the response information AJ indicating the response from the driver DV to the speech output from, for example, the in-vehicle apparatus 3.

Next, in step S119, the output control unit 119 determines whether the response information AJ indicates an unpleasant emotion of the driver DV.

When the output control unit 119 determines that the response information AJ does not indicate an unpleasant emotion of the driver DV (NO in step S119), the processing proceeds to step S125. When the output control unit 119 determines that the response information AJ indicates an unpleasant emotion of the driver DV (YES in step S119), the processing proceeds to step S121.

In step S121, the output control unit 119 instructs the speech output unit 114 to stop the speech output indicating the advice information AD.

Next, in step S123, the output control unit 119 instructs the image output unit 116 to start the image output for the driver DV.

Next, in step S125, the first control unit 11 determines whether the driver DV has stopped driving the vehicle VC. The first control unit 11 determines, for example, that the driver DV has stopped driving the vehicle VC when the smartphone 1 is removed from the holder 350.

When the first control unit 11 determines that the driver DV has not stopped driving the vehicle VC (NO in step S125), the processing returns to step S107. When the first control unit 11 determines that the driver DV has stopped driving the vehicle VC (YES in step S125), the processing ends.

Step S107 corresponds to an example of a “first acquisition step”.

Step S111 corresponds to an example of a “second acquisition step”.

Step S113 corresponds to an example of a “calculation step”.

Step S115 corresponds to an example of an “output step”.

Step S117 corresponds to an example of a “third acquisition step”.

Step S121 corresponds to an example of an “output control step”.

### 7. Configurations and Effects

As described with reference to FIGS. 1 to 7, the smartphone 1 according to the present embodiment includes: the first biological information acquisition unit 111 that acquires the first biological information BJ1 being biological information about the driver DV while driving the vehicle VC; the driving behavior acquisition unit 112 that acquires the driving behavior information QJ indicating the driving behavior of the driver DV; the danger level calculation unit 113 that calculates the driving danger level DR indicating the danger degree while the driver DV is driving the vehicle VC, based on the first biological information BJ1 and the driving behavior information QJ; the speech output unit 114 that performs the speech output for the driver DV, in accordance with the degree of change in the driving danger level DR; the response acquisition unit 115 that acquires the response information AJ indicating the response from the driver DV to the speech output; and the output control unit 119 that controls the speech output unit 114 based on the response information AJ.

Therefore, it is possible to appropriately perform the speech output, since the driving danger level DR indicating the danger degree while the driver DV is driving the vehicle VC is calculated based on the first biological information BJ1 and the driving behavior information QJ, and the speech output for the driver DV is performed in accordance with the degree of change in the driving danger level DR. It is also possible to appropriately control the speech output, since the response information AJ indicating the response from the driver DV to the speech output is acquired and the speech output is controlled based on the response information AJ.

In the smartphone 1, the second biological information acquisition unit 117 is provided that acquires the second biological information BJ2 being biological information about the driver DV before starting to drive the vehicle VC, the health state evaluation unit 118 is provided that evaluates the health state of the driver DV based on the second biological information BJ2 and generates the health rating RS indicating the healthiness degree, and the speech output unit 114 performs the speech output based on the health rating RS and the degree of change in the driving danger level DR.

Therefore, it is possible to appropriately perform the speech output, since the speech output is performed based on the health rating RS indicating the healthiness degree of the driver DV and the degree of change in the driving danger level DR.

In the smartphone 1, the output control unit 119 causes the speech output unit 114 to increase the frequency of performing the speech output, when the health rating RS is equal to or higher than the predetermined value, and after the driving behavior acquisition unit 112 acquires the driving behavior information QJ increasing the driving danger level DR, and then the driving behavior acquisition unit 112 acquires, in the first period, the driving behavior information QJ indicating that the driving behavior has not been improved.

That is, the frequency of performing the speech output is increased when the driver DV is in a healthy state, and the driving behavior information QJ indicating that the driving behavior has not been improved is acquired in the first period, after the driving behavior information QJ increasing the driving danger level DR is acquired. Therefore, it is possible to appropriately increase the frequency of performing the speech output, by appropriately setting the first period.

In the smartphone 1, when the health rating RS is equal to or higher than the predetermined value, and after the driving behavior acquisition unit 112 acquires the driving behavior information QJ increasing the driving danger level DR, and then the driving behavior acquisition unit 112 acquires, in the second period, the driving behavior information QJ indicating that the driving behavior has been improved, the output control unit 119 causes the speech output unit 114 to decrease the frequency of performing the speech output.

That is, the frequency of performing the speech output is decreased when the driver DV is in a healthy state, and the driving behavior information QJ indicating that the driving behavior has been improved is acquired in the second period, after the driving behavior information QJ increasing the driving danger level DR is acquired. Therefore, it is possible to appropriately decrease the frequency of performing the speech output, by appropriately setting the second period.

In the smartphone 1, the speech output unit 114 performs the speech output at the predetermined frequency, in accordance with the degree of change in the driving danger level DR and the travel route traveled by the vehicle VC.

Therefore, it is possible to appropriately adjust the frequency of performing the speech output, since the speech output is performed at the predetermined frequency in accordance with the degree of change in the driving danger level DR and the travel route traveled by the vehicle VC. For example, the frequency of performing the speech output is preferably increased when a plurality of locations are present in proximity to each other where traffic accidents frequently occur on the travel route traveled by the vehicle VC.

In the smartphone 1, the danger level calculation unit 113 generates the reference driving property indicating the property of the driving behavior of the driver, based on the history of the driving behavior information QJ in the past driving by the driver DV; evaluates the variability degree in the reference driving property of the driving behavior indicated by the driving behavior information QJ acquired by the driving behavior acquisition unit 112 in the current driving; and calculates the driving danger level DR indicating the danger degree while the driver DV is driving the vehicle VC, based on the variability degree.

That is, the reference driving property indicating the property of the driving behavior of the driver is generated based on the history of the driving behavior information QJ in the past driving by the driver DV, the variability degree in the reference driving property of the driving behavior indicated by the driving behavior information QJ acquired in the current driving is evaluated; and the driving danger level DR indicating the danger degree while the driver DV is driving the vehicle VC is calculated based on the variability degree. Therefore, it is possible to appropriately calculate the driving danger level DR.

In the smartphone 1, when the response information AJ acquired by the response acquisition unit 115 indicates an unpleasant emotion of the driver DV, the output control unit 119 instructs the speech output unit 114 to stop the speech output or lower the frequency of performing the speech output.

Therefore, it is possible to appropriately control the speech output in accordance with the emotion of the driver DV, since stopping the speech output or lowering the frequency of performing the speech output is instructed when the response information AJ indicates an unpleasant emotion of the driver DV.

In the smartphone 1, the image output unit 116 is provided that performs the image output for the driver DV, in accordance with the degree of change in the driving danger level DR, and the output control unit 119 instructs the image output unit 116 to perform the image output for the driver DV when the response information AJ acquired by the response acquisition unit 115 indicates an unpleasant emotion of the driver DV.

Therefore, it is possible to appropriately control the image output in accordance with the emotion of the driver DV, since performing the image output for the driver DV is instructed when the response information AJ indicates an unpleasant emotion of the driver DV. This is because the image output is less likely to cause an unpleasant emotion of the driver DV in comparison to the speech output.

In the smartphone 1, when the response information AJ acquired by the response acquisition unit 115 indicates an unpleasant emotion of the driver DV and the driving behavior acquisition unit 112 acquires, in the third period, the driving behavior information QJ indicating that the driving behavior has not been improved, the output control unit 119 instructs the speech output unit 114 to perform the speech output a matter that is not directly related to a driving operation of the driver DV.

When the response information AJ indicates an unpleasant emotion of the driver DV and the driving behavior information QJ indicating that the driving behavior has not been improved is acquired in the third period, performing the speech output of a matter that is not directly related to a driving operation of the driver DV is instructed. Therefore, it is possible to appropriately control the speech output in accordance with the emotion of the driver DV, by appropriately setting the third period.

In the smartphone 1, the output control unit 119 instructs the speech output unit 114 to perform the speech output with different content between when the driving danger level DR is increased based on the first biological information BJ1 and when the driving danger level DR is increased based on the driving behavior information QJ.

Therefore, it is possible to appropriately control the speech output, since performing the speech output with different content is instructed when the driving danger level DR is increased based on the first biological information BJ1 and when the driving danger level DR is increased based on the driving behavior information QJ.

The notification control method according to the present embodiment includes: the first acquisition step of acquiring the first biological information BJ1 being biological information about the driver DV while driving the vehicle VC; the second acquisition step of acquiring the driving behavior information QJ indicating the driving behavior of the driver DV; the calculation step of calculating the driving danger level DR indicating the danger degree while the driver DV is driving the vehicle VC, based on the first biological information BJ1 and the driving behavior information QJ; the output step of performing the speech output for the driver DV, in accordance with the degree of change in the driving danger level DR; the third acquisition step of acquiring the response information AJ indicating the response from the driver DV to the speech output; and the output control step of controlling the speech output based on the response information AJ.

Thus, the notification control method according to the present embodiment exhibits operations and effects similar to those of the smartphone 1 according to the present embodiment.

The first control program PG1 according to the present embodiment causes the first processor 11A included in the smartphone 1 to function as: the first biological information acquisition unit 111 that acquires the first biological information BJ1 being biological information about the driver DV while driving the vehicle VC; the driving behavior acquisition unit 112 that acquires the driving behavior information QJ indicating the driving behavior of the driver DV; the danger level calculation unit 113 that calculates the driving danger level DR indicating the danger degree while the driver DV is driving the vehicle VC, based on the first biological information BJ1 and the driving behavior information QJ; the speech output unit 114 that performs the speech output for the driver DV, in accordance with the degree of change in the driving danger level DR; the response acquisition unit 115 that acquires the response information AJ indicating the response from the driver DV to the speech output; and the output control unit 119 that controls the speech output unit 114 based on the response information AJ.

Thus, the first control program PG1 according to the present embodiment exhibits operations and effects similar to those of the smartphone 1 according to the present embodiment.

### 8. Other Embodiments

The embodiment described above is a preferred embodiment of the present invention. However, the present invention is not limited to the embodiment described above, and can be implemented with a variety of modifications within the scope or the spirit of the present invention.

In the present embodiment, a case is described in which the “notification control apparatus” is implemented as the smartphone 1, but the embodiment is not limited thereto. The “notification control apparatus” may, for example, be implemented as a personal computer, a tablet apparatus, a server apparatus, or the like. At least part of the “notification control apparatus” may be implemented as the ECU 31 of the in-vehicle apparatus 3.

In the present embodiment, a case is described in which the smartwatch 2 generates the biological information about the driver DV, but the embodiment is not limited thereto. The “device” that generates the biological information about the driver DV may be worn by the driver DV and may generate the biological information BJ.

For example, the “device” that generates the biological information about the driver DV may be worn on an arm, the head, a leg, or the like of the driver DV. For example, the “device” may be smart innerwear attached to innerwear worn by a user U. For example, the “device” may be a sensing device attached to the skin, or inside the body of the user U.

In the present embodiment, a case is described in which the speech output unit 114 outputs speech indicating the advice information AD from the speaker 16, but the embodiment is not limited thereto. The speech output unit 114 may output the speech indicating the advice information AD from at least one of the left speaker 333a or the right speaker 333b of the vehicle VC.

In the present embodiment, a case is described in which the image output unit 116 displays an image indicating the advice information AD on the first touch panel 14, but the embodiment is not limited thereto. The image output unit 116 may display the image indicating the advice information AD on a fourth touch panel 44 included in the in-vehicle apparatus 3.

For example, each of the configurations of the smartwatch 2 shown in FIG. 2, the in-vehicle apparatus 3 shown in FIG. 3, and the smartphone 1 shown in FIG. 5 indicates a functional configuration, and the specific embodiment thereof is not particularly limited. In other words, hardware individually corresponding to the respective functional units does not necessarily need to be implemented, and it is possible to adopt a configuration in which one processor executes a program to realize functions of a plurality of functional units. Part of functions realized by software in the above embodiments may be realized by hardware or part of functions realized by hardware may be realized by software.

The units of processing of the flowchart shown in FIG. 7 are divided in accordance with the main processing content in order to facilitate understanding of the processing of the smartphone 1, but the present invention is not limited by the division method, naming, or the like of the units of processing shown in the flowchart of FIG. 7. The processing of the smartphone 1 can be further divided into more units of processing in accordance with the content of the processing, or one unit of processing can be further divided to include more processing. The processing order of the above flowchart is also not limited to the illustrated example.

### 9. Configurations Supported by the Above Embodiments

The above embodiments are specific examples of the following configurations.

### Configuration 1

A notification control apparatus includes: a first biological information acquisition unit configured to acquire first biological information being biological information about a driver while driving a vehicle; a driving behavior acquisition unit configured to acquire driving behavior information indicating driving behavior of the driver; a danger level calculation unit configured to calculate a driving danger level indicating a danger degree while the driver is driving the vehicle, based on the first biological information and the driving behavior information; a speech output unit configured to perform speech output for the driver, in accordance with a degree of change in the driving danger level; a response acquisition unit configured to acquire response information indicating a response from the driver to the speech output; and an output control unit configured to control the speech output unit, based on the response information.

The notification control apparatus of Configuration 1 can appropriately perform the speech output, since the driving danger level indicating the danger degree while the driver is driving the vehicle is calculated based on the first biological information and the driving behavior information, and the speech output for the driver is performed in accordance with the degree of change in the driving danger level. It is also possible to appropriately control the speech output, since the response information indicating the response from the driver to the speech output is acquired and the speech output is controlled based on the response information.

### Configuration 2

The notification control apparatus according to Configuration 1 includes: a second biological information acquisition unit configured to acquire second biological information being biological information about the driver before starting to drive the vehicle; and a health state evaluation unit configured to evaluate a health state of the driver and generate a health rating indicating a healthiness degree, based on the second biological information, in which the speech output unit performs the speech output, based on the health rating and the degree of change in the driving danger level.

The notification control apparatus of Configuration 2 can appropriately perform the speech output, since the speech output is performed based on the health rating indicating the healthiness degree of the driver and the degree of change in the driving danger level.

### Configuration 3

The notification control apparatus according to Configuration 2, in which when the health rating is equal to or higher than a predetermined value, and after the driving behavior acquisition unit acquires the driving behavior information increasing the driving danger level, and then the driving behavior acquisition unit acquires, in a first period, driving behavior information indicating that the driving behavior has not been improved, the output control unit causes the speech output unit to increase a frequency of performing the speech output.

The notification control apparatus of Configuration 3 can appropriately increase the frequency of performing the speech output, by appropriately setting the first period.

### Configuration 4

The notification control apparatus according to Configuration 2 or 3, in which when the health rating is equal to or higher than a predetermined value, and after the driving behavior acquisition unit acquires the driving behavior information increasing the driving danger level, and then the driving behavior acquisition unit acquires, in a second period, driving behavior information indicating that the driving behavior has been improved, the output control unit causes the speech output unit to decrease a frequency of performing the speech output.

The notification control apparatus of Configuration 4 can appropriately decrease the frequency of performing the speech output, by appropriately setting the second period.

### Configuration 5

The notification control apparatus according to any one of Configurations 1 to 4, in which the speech output unit performs the speech output at a predetermined frequency, in accordance with the degree of change in the driving danger level and a travel route traveled by the vehicle.

The notification control apparatus of Configuration 5 can appropriately adjust the frequency of performing the speech output, since the speech output is performed at the predetermined frequency in accordance with the degree of change in the driving danger level and the travel route traveled by the vehicle.

### Configuration 6

The notification control apparatus according to any one of Configurations 1 to 5, in which the danger level calculation unit: generates a reference driving property indicating a property of the driving behavior of the driver, based on a history of the driving behavior information in past driving by the driver; evaluates a variability degree in the reference driving property of the driving behavior indicated by the driving behavior information acquired by the driving behavior acquisition unit in current driving; and calculates the driving danger level indicating the danger degree while the driver is driving the vehicle, based on the variability degree.

The notification control apparatus of Configuration 6 can appropriately calculate the driving danger level.

### Configuration 7

The notification control apparatus according to any one of Configurations 1 to 6, in which the output control unit instructs the speech output unit to stop the speech output or lower a frequency of performing the speech output, when the response information acquired by the response acquisition unit indicates an unpleasant emotion of the driver.

The notification control apparatus of Configuration 7 can appropriately control the speech output in accordance with the emotion of the driver, since stopping the speech output or lowering the frequency of performing the speech output is instructed when the response information indicates an unpleasant emotion of the driver.

### Configuration 8

The notification control apparatus according to any one of Configurations 1 to 7 includes an image output unit configured to perform image output for the driver, in accordance with the degree of change in the driving danger level, in which the output control unit instructs the image output unit to perform the image output for the driver, when the response information acquired by the response acquisition unit indicates an unpleasant emotion of the driver.

The notification control apparatus of Configuration 8 can appropriately control the image output in accordance with the emotion of the driver, since performing the image output for the driver is instructed when the response information indicates an unpleasant emotion of the driver. This is because the image output is less likely to cause an unpleasant emotion of the driver in comparison to the speech output.

### Configuration 9

The notification control apparatus according to any one of Configurations 1 to 8, in which when the response information acquired by the response acquisition unit indicates an unpleasant emotion of the driver and the driving behavior acquisition unit acquires, in a third period, the driving behavior information indicating that the driving behavior has not been improved, the output control unit instructs the speech output unit to perform speech output of a matter that is not directly related to a driving operation of the driver.

The notification control apparatus of Configuration 9 can appropriately control the speech output in accordance with the emotion of the driver, by appropriately setting the third period.

### Configuration 10

The notification control apparatus according to any one of Configurations 1 to 9, in which the output control unit instructs the speech output unit to perform speech output with different content between when the driving danger level is increased based on the first biological information and when the driving danger level is increased based on the driving behavior information.

The notification control apparatus of Configuration 10 can appropriately control the speech output, since performing the speech output with different content is instructed when the driving danger level is increased based on the first biological information and when the driving danger level is increased based on the driving behavior information.

### Configuration 11

A notification control method includes: a first acquisition step of acquiring first biological information being biological information about a driver while driving a vehicle; a second acquisition step of acquiring driving behavior information indicating driving behavior of the driver; a calculation step of calculating a driving danger level indicating a danger degree while the driver is driving the vehicle, based on the first biological information and the driving behavior information; an output step of performing speech output for the driver, in accordance with a degree of change in the driving danger level; a third acquisition step of acquiring response information indicating a response from the driver to the speech output; and an output control step of controlling the speech output unit, based on the response information.

The notification control method of Configuration 11 has a configuration similar to that of the notification control apparatus of Configuration 1, and thus exhibits operations and effects similar to those of the notification control apparatus of Configuration 1.

### Configuration 12

A non-transitory computer readable storage medium storing a notification control program causes a processor included in a notification control apparatus to function as: a first biological information acquisition unit configured to acquire first biological information being biological information about a driver while driving a vehicle; a driving behavior acquisition unit configured to acquire driving behavior information indicating driving behavior of the driver; a danger level calculation unit configured to calculate a driving danger level indicating a danger degree while the driver is driving the vehicle, based on the first biological information and the driving behavior information; a speech output unit configured to perform speech output for the driver, in accordance with a degree of change in the driving danger level; a response acquisition unit configured to acquire response information indicating a response from the driver to the speech output; and an output control unit configured to control the speech output unit, based on the response information.

The storage medium of Configuration 12 has a configuration similar to that of the notification control apparatus of Configuration 1, and thus exhibits operations and effects similar to those of the notification control apparatus of Configuration 1.

### REFERENCE SIGNS LIST

100 notification control system
1 smartphone (notification control apparatus)
11 first control unit
11A first processor (processor)
11B first memory
110 information storage unit
111 first biological information acquisition unit
112 driving behavior acquisition unit
113 danger level calculation unit
114 speech output unit
115 response acquisition unit
116 image output unit
117 second biological information acquisition unit
118 health state evaluation unit
119 output control unit
16 speaker
17 microphone
14 first touch panel
2 smartwatch
25 biological information generation unit
3 in-vehicle apparatus
31 ECU
35 driving information generation unit
AB driving ability
AB1 planning ability
AB2 perception ability
AB3 attention ability
AB4 judgment ability
AB5 operation ability
AD advice information
AJ response information
BJ biological information
BJ1 first biological information
BJ11 first sleep information
BJ12 first exercise information
BJ13 first stress information
BJ2 second biological information
BJ21 second sleep information
BJ22 second exercise information
BJ23 second stress information
DV driver
PG1 first control program (notification control program)
QJ driving information
RS health rating
RS1 planning ability rating
RS2 perception ability rating
RS3 attention ability rating
RS4 judgment ability rating
RS5 operation ability rating
VC vehicle

1. A notification control apparatus, comprising:
a first biological information acquisition unit configured to acquire first biological information being biological information about a driver while driving a vehicle;
a driving behavior acquisition unit configured to acquire driving behavior information indicating driving behavior of the driver;
a danger level calculation unit configured to calculate a driving danger level indicating a danger degree while the driver is driving the vehicle, based on the first biological information and the driving behavior information;
a speech output unit configured to perform speech output for the driver, in accordance with a degree of change in the driving danger level;
a response acquisition unit configured to acquire response information indicating a response from the driver to the speech output; and
an output control unit configured to control the speech output unit, based on the response information.

2. The notification control apparatus according to claim 1, comprising:
a second biological information acquisition unit configured to acquire second biological information being biological information about the driver before starting to drive the vehicle; and
a health state evaluation unit configured to evaluate a health state of the driver and generate a health rating indicating a healthiness degree, based on the second biological information, wherein
the speech output unit performs the speech output, based on the health rating and the degree of change in the driving danger level.

3. The notification control apparatus according to claim 2, wherein when the health rating is equal to or higher than a predetermined value, and after the driving behavior acquisition unit acquires the driving behavior information increasing the driving danger level, and then the driving behavior acquisition unit acquires, in a first period, driving behavior information indicating that the driving behavior has not been improved, the output control unit causes the speech output unit to increase a frequency of performing the speech output.

4. The notification control apparatus according to claim 2, wherein when the health rating is equal to or higher than a predetermined value, and after the driving behavior acquisition unit acquires the driving behavior information increasing the driving danger level, and then the driving behavior acquisition unit acquires, in a second period, driving behavior information indicating that the driving behavior has been improved, the output control unit causes the speech output unit to decrease a frequency of performing the speech output.

5. The notification control apparatus according to claim 1, wherein the speech output unit performs the speech output at a predetermined frequency, in accordance with the degree of change in the driving danger level and a travel route traveled by the vehicle.

6. The notification control apparatus according to claim 1, wherein
the danger level calculation unit:
generates a reference driving property indicating a property of the driving behavior of the driver, based on a history of the driving behavior information in past driving by the driver;
evaluates a variability degree in the reference driving property of the driving behavior indicated by the driving behavior information acquired by the driving behavior acquisition unit in current driving; and
calculates the driving danger level indicating the danger degree while the driver is driving the vehicle, based on the variability degree.

7. The notification control apparatus according to speech output unit to stop the speech output or lower a frequency of performing the speech output, when the response information acquired by the response acquisition unit indicates an unpleasant emotion of the driver.

8. The notification control apparatus according to claim 1, comprising an image output unit configured to perform image output for the driver, in accordance with the degree of change in the driving danger level, wherein
the output control unit instructs the image output unit to perform the image output for the driver, when the response information acquired by the response acquisition unit indicates an unpleasant emotion of the driver.

9. The notification control apparatus according to claim 1, wherein when the response information acquired by the response acquisition unit indicates an unpleasant emotion of the driver and the driving behavior acquisition unit acquires, in a third period, the driving behavior information indicating that the driving behavior has not been improved, the output control unit instructs the speech output unit to perform speech output of a matter that is not directly related to a driving operation of the driver.

10. The notification control apparatus according to speech output unit to perform speech output with different content between when the driving danger level is increased based on the first biological information and when the driving danger level is increased based on the driving behavior information.

11. A notification control method, comprising:
a first acquisition step of acquiring first biological information being biological information about a driver while driving a vehicle;
a second acquisition step of acquiring driving behavior information indicating driving behavior of the driver;
a calculation step of calculating a driving danger level indicating a danger degree while the driver is driving the vehicle, based on the first biological information and the driving behavior information;
an output step of performing speech output for the driver, in accordance with a degree of change in the driving danger level;
a third acquisition step of acquiring response information indicating a response from the driver to the speech output; and
an output control step of controlling the speech output, based on the response information.

12. A non-transitory computer readable storage medium storing a notification control program for causing a processor included in a notification control apparatus to function as:
a first biological information acquisition unit configured to acquire first biological information being biological information about a driver while driving a vehicle;
a driving behavior acquisition unit configured to acquire driving behavior information indicating driving behavior of the driver;
a danger level calculation unit configured to calculate a driving danger level indicating a danger degree while the driver is driving the vehicle, based on the first biological information and the driving behavior information;
a speech output unit configured to perform speech output for the driver, in accordance with a degree of change in the driving danger level;
a response acquisition unit configured to acquire response information indicating a response from the driver to the speech output; and
an output control unit configured to control the speech output unit, based on the response information.
